(19) United States
(12) Patent Application Publication
(10) Pub. No.: US 2023/0218844 A1
(43) Pub. Date: Jul. 13, 2023

(54) Systems And Methods For Therapy Cessation Diagnoses

(71) Applicant: ResMed Pty Ltd, Bella Vista NSW (AU)

(72) Inventors: Jamie Graeme Wehbeh, Bella Vista NSW (AU); Liam Holley, Sydney (AU); Nicolous Carr, Bella Vista NSW (AU); Genevieve Claire Madafiglio, Sydney (AU)

(21) Appl. No.: 17/907,022

(22) PCT Filed: Sep. 28, 2021
(86) PCT No.: PCT/IB2021/058854

§ 371 Date: Sep. 22, 2022

Related U.S. Application Data
(60) Provisional application No. 63/198,126, filed on Sep. 30, 2020.

(51) Int. Cl. A61M 16/00; A61M 16/06; A61M 16/22

(52) U.S. Cl. CPC A61M 16/024; A61M 16/0605; A61M 16/22; A61M 2205/3344; A61M 2016/0039; A61M 2230/62

(57) ABSTRACT

A method includes applying, via a respiratory therapy system, initial therapy settings for a user during a first sleep session in which the user uses the respiratory therapy system. First physiological data, which is received from one or more sensors, is generated during the first sleep session. Modified therapy settings are applied, via the respiratory therapy system, during a second sleep session of the user. Second physiological data is received from the one or more sensors. The second physiological data is generated by the one or more sensors during the second sleep session. A set of sleep-related parameters is determined based on changes between the first physiological data and the second physiological data. One or more of a recommended therapy or recommended therapy settings is determined based on the set of sleep-related parameters.

### CROSS-REFERENCE TO RELATED APPLICATIONS

This application claims the benefit of and priority to U.S. Provisional Patent Application No. 63/198,126, filed Sep. 30, 2020, which is hereby incorporated by reference herein in its entirety.

### TECHNICAL FIELD

The present disclosure relates generally to systems and methods for determining a recommended therapy for a user, and more particularly, to systems and methods for determining the recommended therapy based at least in part on changes in physiological data of the user received from two respective sleep sessions.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), chest wall disorders, and insomnia. Many of these disorders can be treated using a respiratory therapy system, while others may be treated using a different technique. These disorders are often diagnosed through a sleep study, and a therapy is prescribed based on the results. However, the prescribed therapy type and/or one or more parameters for the therapy are often selected based on broad generalizations, and may not be suited to the particular individual. The present disclosure is directed to solving these and other problems.

### SUMMARY

According to some implementations of the present disclosure, a method includes applying, via a respiratory therapy system, initial therapy settings for a user during a first sleep session in which the user uses the respiratory therapy system. The method further includes receiving, from one or more sensors, first physiological data generated during the first sleep session. Modified therapy settings are applied, via the respiratory therapy system, during a second sleep session of the user. The second sleep session is subsequent to the first sleep session, and the modified therapy settings are different than the initial therapy settings. Second physiological data is received from the one or more sensors. The second physiological data is generated by the one or more sensors during the second sleep session. A set of sleep-related parameters is determined based on changes between the first physiological data and the second physiological data. One or more of a recommended therapy or recommended therapy settings is determined based on the set of sleep-related parameters.

According to some other implementations of the present disclosure, a system includes a respiratory therapy system having a respiratory device configured to supply pressurized air. The respiratory therapy system further has a user interface coupled to the respiratory device via a conduit, the user interface being configured to engage a user and aid in directing the supplied pressurized air to an airway of the user. The system further includes a memory storing machine-readable instructions, and a control system including one or more processors configured to execute the machine-readable instructions. The machine-readable instructions are executed to apply initial therapy settings for the user during a first sleep session in which the user uses the respiratory therapy system, and to receive first physiological data generated by one or more sensors during the first sleep session. The machine-readable instructions are further executed to apply modified therapy settings during a second sleep session of the user. The second sleep session is subsequent to the first sleep session, and the modified therapy settings are different than the initial therapy settings. The machine-readable instructions are further executed to receive second physiological data generated by the one or more sensors during the second sleep session, and to determine a set of sleep-related parameters based on changes between the first physiological data and the second physiological data. The machine-readable instructions are further executed to determine recommended therapy settings based on the set of sleep-related parameters.

According to some other implementations of the present disclosure, a method is directed to supplying, via a respiratory therapy system, an initial air flow to a user during a first sleep session in which the user uses the respiratory therapy system. First physiological data, which is received from one or more sensors, is generated during the first sleep session. The initial air flow is decreased, via the respiratory therapy system, to a modified air flow during a second sleep session of the user. The second sleep session is subsequent to the first sleep session. Second physiological data, which is received from the one or more sensors, is generated by the one or more sensors during the second sleep session. The second physiological data is indicative only of breathing of the user. A set of sleep-related parameters is determined based on changes between the first physiological data and the second physiological data. Recommended therapy settings are determined based on the set of sleep-related parameters.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of exemplary embodiments together with reference to the accompanying drawings. These drawings depict only exemplary embodiments, and are therefore not to be considered as limitations on the scope of the various embodiments or claims.

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure; and

FIG. 5 is a process flow diagram for a method for recommending a therapy to a user, according to some implementations of the present disclosure.

FIG. 6 is a process flow diagram for additional steps of recommending the therapy of

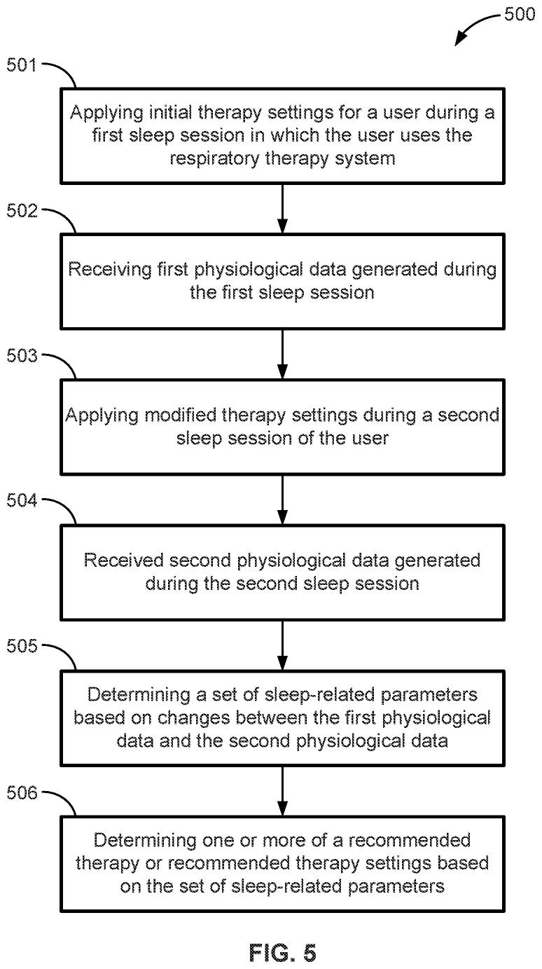

FIG. 5, according to some implementations of the present disclosure.

FIG. 7A is a side view illustrating a user inhaling air through a mask.

FIG. 7B is a side view illustrating the user of FIG. 7A exhaling air through the mask.

While the invention is susceptible to various modifications and alternative forms, specific implementations have been shown by way of example in the drawings and will be described in further detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Various embodiments are described with reference to the attached figures, where like reference numerals are used throughout the figures to designate similar or equivalent elements. The figures are not drawn to scale and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The various embodiments are not limited by the illustrated ordering of acts or events, as some acts may occur in different orders and/or concurrently with other acts or events. Furthermore, not all illustrated acts or events are required to implement a methodology in accordance with the present invention.

Elements and limitations that are disclosed, for example, in the Abstract, Summary, and Detailed Description sections, but not explicitly set forth in the claims, should not be incorporated into the claims, singly, or collectively, by implication, inference, or otherwise. For purposes of the present detailed description, unless specifically disclaimed, the singular includes the plural and vice versa. The word “including” means “including without limitation.” Moreover, words of approximation, such as “about,” “almost,” “substantially,” “approximately,” “generally,” and the like, can be used herein to mean “at,” “near,” or “nearly at,” or “within 3-5% of,” or “within acceptable manufacturing tolerances,” or any logical combination thereof, for example.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as central apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

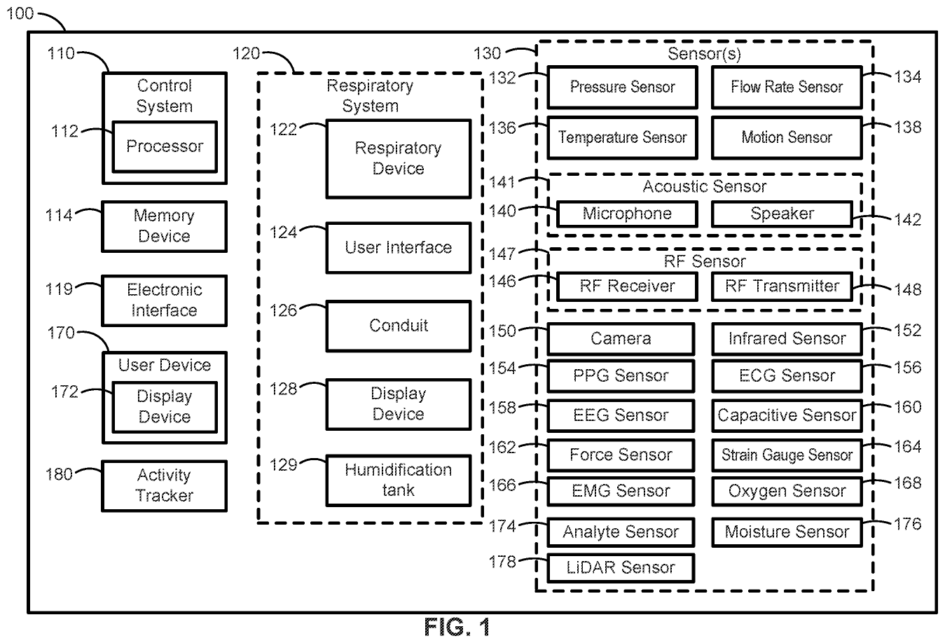

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory system 120, an activity tracker 180, or any combination thereof.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, a portion (e.g., a housing) of the respiratory system 120, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of a respiratory device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia, an employment status of the user, an educational status of the user, a socioeconomic status of the user, or any combination thereof. The medical information can include, for example, including indicative of one or more medical conditions associated with the user, medication usage by the user, or both. The medical information data can further include a multiple sleep latency test (MSLT) test result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory pressure therapy device 122 (referred to herein as respiratory device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 liters/minute (“L/min”) and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

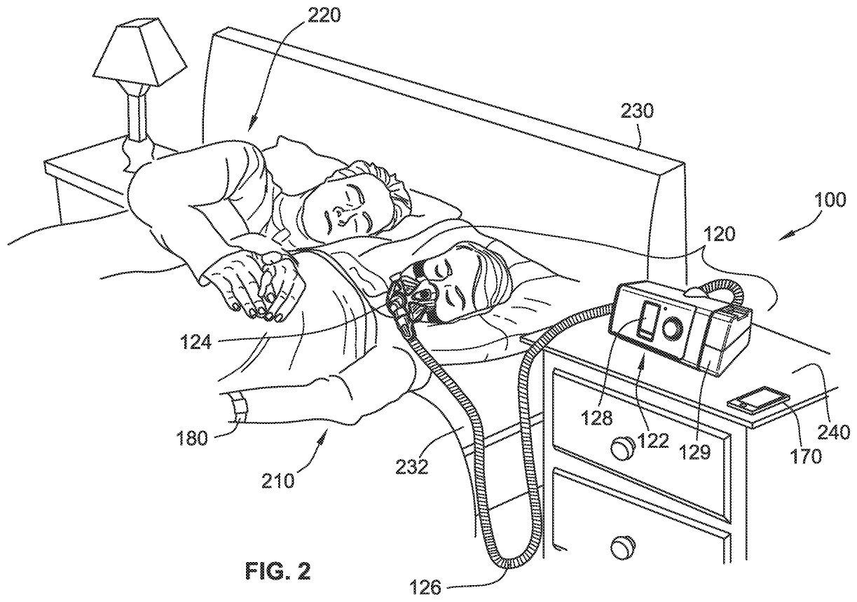

As shown in FIG. 2, in some implementations, the user interface 124 is a full face mask that covers the nose and mouth of the user. Alternatively, the user interface 124 may be an ultra-compact full face mask that provides air to the nose and mouth of the user. Alternatively, the user interface 124 can be a nasal mask that provides air to the nose of the user, or a nasal pillow or cradle mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 is a mouthpiece (e.g., a night guard mouthpiece molded to conform to the user's teeth, a mandibular repositioning device, etc.) for directing pressurized air into the mouth of the user.

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score (also referred to as a myAir score), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory system 120 and a bed partner 220 are located in a bed 230 and are laying on a mattress 232. The user interface 124 (e.g., a full face mask) can be worn by the user 210 during a sleep session. The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, a temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a Light Detection and Ranging (LiDAR) sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory device 122, the use interface 124, the conduit 126, or the user device 170.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a face mask that covers the nose and mouth of the user 210, the analyte sensor 174 can be positioned within the face mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the face mask (in implementations where the user interface 124 is a face mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The LiDAR sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, or any combination thereof. For example, the microphone 140 and speaker 142 is integrated in and/or coupled to the user device 170 and the pressure sensor 130 and/or flow rate sensor 132 are integrated in and/or coupled to the respiratory device 122. In some implementations, at least one of the one or more sensors 130 is not coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The activity tracker 180 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 180 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 180 is a wearable device that can be worn by the user, such as a smartwatch, a wristband, a ring, or a patch. For example, referring to FIG. 2, the activity tracker 180 is worn on a wrist of the user 210. The activity tracker 180 can also be coupled to or integrated a garment or clothing that is worn by the user. Alternatively still, the activity tracker 180 can also be coupled to or integrated in (e.g., within the same housing) the user device 170. More generally, the activity tracker 180 can be communicatively coupled with, or physically integrated in (e.g., within a housing), the control system 110, the memory 114, the respiratory system 120, and/or the user device 170.

While the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory system 120, at least one of the one or more sensors 130, and the user device 170. Thus, various systems can be formed using any portion or portions of the components shown and described herein and/or in combination with one or more other components.

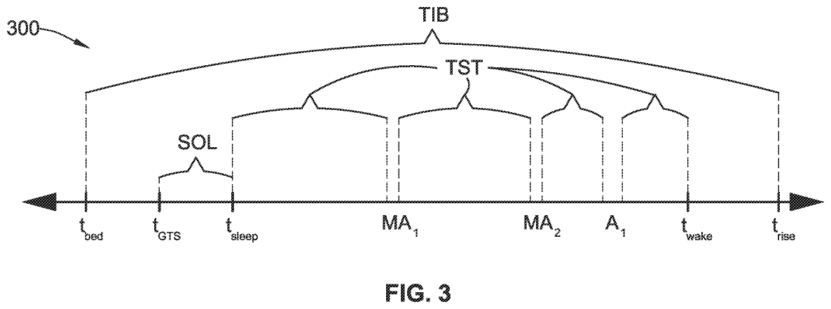

As used herein, a sleep session can be defined in a number of ways based on, for example, an initial start time and an end time. Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ).

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time tₑₐₖₑ, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time -rise is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial tb_{ed} and the final trice. In some implementations, the final wake-up time t_{wake} and/or the final rising time -rise that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛe. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

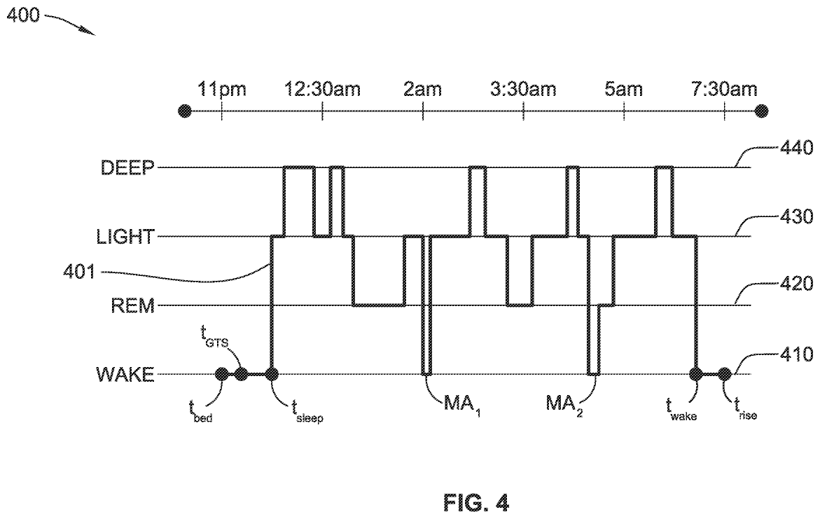

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (teed), the go-to-sleep time (t_{G}rs), the initial sleep time (tₛₗₑₑₚ), one or more first micro-awakenings (e.g., MA₁ and MA₂), the wake-up time (t_{wake}), the rising time (trise), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights), data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory device 122, data indicative of the user donning the user interface 124, etc.), or any combination thereof.

Generally, users that already uses one type of therapy, such as CPAP, would benefit from knowing what they would experience if that therapy was stopped or diminished. This “what if” knowledge is extremely beneficial to empower the user into continuing the therapy, modifying the therapy, or changing the therapy. For example, a user might experience increased apnea events if CPAP therapy was stopped, which, in turn, may help the user continue the CPAP with increased commitment. Without knowing the consequences of stopping or decreasing the therapy, the user might simply give up on the therapy or give it a lower priority that will essentially decrease benefits otherwise associated with the therapy.

According to one non-limiting example in which the user is using CPAP therapy, pressurized air flow is shut off during a sleep session. The shut off can occur during an optimal time based on the user's sleep state, for example, to avoid waking up the user. While the pressurized air flow is shut off, respiration of the user is measured using the same sensors as those measuring the respiration of the user when the user is using the CPAP therapy. The measured respiration of the user, which represents a sleep-related parameter, is then compared between (a) measured respiration during a regular sleep session using CPAP therapy, and (b) measured respiration during a sleep session in which the shut off occurs. Based on the changes in the measured respiration of the two different sleep sessions, the user is provided with a recommended therapy and/or recommended therapy settings, such as continuing CPAP therapy without any changes, continuing CPAP therapy with decreased pressurized air flow, or discontinuing CPAP therapy and pursuing a surgical procedure.

Depending on the user, to avoid potential breathing problems, instead of completely shutting off the pressurized air flow a low flow rate is provided. For example, if a full face mask is used by the user, completely shutting off the pressurized air flow may result in unhealthy build-up of carbon dioxide in the mask. To avoid this type of potential problem, the low flow rate (e.g., a control flow rate) is provided either alone or in combination with one or more vents in the mask (as described below in more detail).

Accordingly, some benefits of the present disclosure provide methods and systems configured to adjust a therapy or therapy settings to achieve a desired health goal of a user. The method or system can monitor health occurrences, such as apnea, over time and provide feedback to the user on whether the user can cease therapy, change therapy, or adjust therapy settings. For example, the feedback may provide an assessment of whether the CPAP therapy works well, if something else occurs that changes the CPAP therapy, or whether the user has been misdiagnosed and is using the wrong therapy.

In accordance to one example, a user is initially overweight while using the CPAP therapy. Over time, the user loses weight, which might cause the user to safely discontinue the CPAP therapy, reduce therapy settings (e.g., reduce flow from flow generator) accordingly, or see less intrusive therapy.

Referring back to FIG. 2, in accordance with another non-limiting example, the system 100 is directed to providing a PAP therapy. The system 100 includes a flow generator in the form of the respiratory device 122, a mask system in the form of the user interface 124, and the conduit 126 that connects the flow generator and the mask system. The system includes at least one sensor (e.g., sensor 130 of FIG. 1) that determines whether the user 210 using the system 100 has experienced, for example, a sleep apnea event, such as obstructive apneas, central apneas, hypopneas, respiratory-effort related arousals, flow limitation, oxygen desaturation, snoring, etc..

In further accordance with the non-limiting example described above, the system 100 is configured to perform a method for determining that the user 210 using the system 100 has experienced the apnea event. The method for determining the apnea occurrence includes selectively decreasing air pressure and/or flow rate delivered to the user 210, using the system 100 to allow the user's own breathing to be monitored. Thus, the apnea occurrence can be detected.

The system 100 is further configured to provide an indication to the user 210 of the apnea occurrence and whether therapy can be discontinued. If continued therapy is required, the system 100 provides appropriate indications to the user 210. The system 100 can provide indications of health improvements over time, such as decreases in apnea rates over time as an indicator of therapy success and/or benefit.

Weaning a user off therapy provides insight into how the user may feel. Optionally, a custom schedule is provided to the user for determining whether continues to benefit from the therapy. The schedule determines what sleep stages or durations should be selected for testing modified therapy settings. For example, the schedule determines whether the modified therapy settings should be applied over the course of days, weeks, or months, with the end goal being to have sufficient physiological data from a test sleep session (e.g., a second sleep session) for determining the user should change the therapy or the therapy settings. Based on the results of the test sleep session, the user might be recommended to try an alternative sleep temperature or an alternative air pressure for CPAP therapy.

Referring to FIG. 5, a method 500 for determining a recommended therapy for a user according to some implementations of the present disclosure is illustrated. One or more steps or aspects of the method 500 can be implemented using any portion or aspect of the system 100 described herein.

Step 501 of the method 500 includes applying initial therapy settings for a user during a first sleep session in which the user uses the respiratory therapy system (e.g., the respiratory system 120 illustrated in FIG. 2). The initial therapy settings include one or more settings related to the therapy that is currently being pursued by the user, such as the respiratory pressure therapy described above in reference to FIG. 1. The initial therapy settings include, for example, pressure settings of the respiratory device 122 (illustrated in FIG. 2), such as air flow rate or air pressure of supplied pressurized air to airway of the user 210 (illustrated in FIG. 2). The air flow rate is monitored, for example, using the flow rate sensor 134 (illustrated in FIG. 1). The air pressure is monitored, for example, using the pressure sensor 132 (illustrated in FIG. 1).

The first sleep session includes, for example, a first portion of a first day (e.g., a Monday) before the user begins the first sleep session, and a second portion of the first day after the user begins the first sleep session. A first portion of first physiological data is associated with the first sleep session. If the first sleep session extends into the next day (e.g., a Tuesday) before ending, the first physiological data for the first sleep session will also be associated with a portion of the second day. In this example, the first portion of the first physiological data can be associated with any portion of the first sleep session (e.g., 10% of the first sleep session, 25% of the first sleep session, 50% of the first sleep session, 100% of the first sleep session, etc.).

In some implementations, the first sleep session is and includes a time period that is prior to the next immediately successive sleep session following the first sleep session. In such implementations, at least one of the one or more sensors 130 generates activity-related physiological data associated with the user subsequent to the first sleep session. For example, the first physiological data associated with the first sleep session can be generated or obtained from a first one or a first group of the one or more sensors 130 described herein, and the activity-related physiological data generated subsequent to the first sleep session can be generated or obtained from a second one or a second group of the one or more sensors 130. For example, the first physiological data can be generated or obtained by a first sensor that is coupled to or integrated in the respiratory system 120 described herein while the activity-related physiological data can be generated or obtained by a second sensor is that is coupled to or integrated in the user device 170 or the activity tracker 180. The activity-related physiological data can be used to determine an activity measurement associated with the user, such as, for example, number of steps, a distance traveled, a number of steps climbed, a duration of physical activity, a type of physical activity, an intensity of physical activity, time spent standing, a respiration rate, an average respiration rate, a resting respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), a measure of venous dilation, or artery tone, or any combination thereof.

Step 502 of the method 500 includes receiving first physiological data associated with a user during the first sleep session. The first physiological data can be generated by, and received from, one or more of the sensors 130 (FIG. 1) described herein. The received first physiological data can indicative of one or more physiological parameters such as, for example, movement, heart rate, heart rate variability, cardiac waveform, respiration rate, respiration rate variability, respiration depth, a tidal volume, an inspiration amplitude, an expiration amplitude, an inspiration-expiration ratio, perspiration, temperature (e.g., ambient temperature, body temperature, core body temperature, surface temperature, etc.), blood oxygenation, photoplethysmography, pulse transmit time, blood pressure, or any combination thereof. The first physiological data can be received from at least one of the one or more sensors 130 by, for example, the electronic interface 119 and/or the user device 170 described herein, and stored in the memory 114 (FIG. 1). The first physiological data can be received by the electronic interface 119 or the user device 170 from at least one of the one or more sensors 130 either directly or indirectly (e.g., with one or more intermediaries).

Step 503 of the method 500 includes applying modified therapy settings during a second sleep session of the user. The modified therapy settings include any settings that are changed relative to the initial therapy settings. For example, modified therapy settings include reducing or eliminating the air flow rate or air pressure of supplied pressurized air to the airway of the user 210 (illustrated in FIG. 2), or changing a sleep temperature to an alternative sleep temperature.

According to one example, the modified therapy settings are determined such that an optimal smooth transition of implemented changes occurs. In other words, for example, a dramatic change between the initial therapy settings and the modified therapy settings might cause the user to wake up. As such, the modified therapy settings may be applied, for example, at the beginning of the second sleep session so that a transition is not introduced. According to another example, the modified therapy settings may be applied at the onset of different sleep states to determine if the user has REM dominant apneas. According to yet another example, the modified therapy settings are applied based on the user's historical data to see where the user's AHI has been the highest, and, based on that analysis, one or more sleep states are selected during the second sleep session to determine the occurrence of a health occurrence, such as an apnea occurrence.

The second sleep session is subsequent to the first sleep session. In some implementations, the second sleep session is the next immediately successive sleep session following the first sleep session (e.g., the first sleep session begins Monday night and ends Tuesday morning and the second sleep session beings on the same Tuesday evening and ends the following Wednesday morning). In other implementations, there are one or more additional sleep sessions between the first sleep session and the second sleep session. The second sleep session differs from the first sleep session in that the user is using modified therapy settings during the second sleep session.

The first sleep session and/or the second sleep session can be a diagnostic sleep session. A diagnostic sleep session is generally a sleep session in which one or more of the sensors 130 are used to diagnose the user or perform a sleep study. For example, the diagnostic sleep session can be an in-home diagnosis sleep session where the user is provided one or more of a pulse oximeter (e.g., a fingertip pulse oximeter) for measuring blood oxygen saturation and one or additional sensors for measuring airflow, respiratory effort, cardiac data, etc. As another example, the diagnostic sleep session can be a polysomnogram (PSG) test performed in a lab with a supervising technician or specialist (e.g., using the ECG sensor 156, the EEG sensor 158, an EMG sensor 166, an EOG sensor, or any combination thereof). In some implementations, the first sleep session is a first type of diagnostic sleep session (e.g., an in-home session) and the second sleep session is a second type of diagnostic sleep session (e.g., a PSG test session). Alternatively, the first sleep session can be a diagnostic sleep session and the second sleep session is not a diagnostic sleep session.

Optionally, the second sleep session is determined based on historical data of the user. For example, the historical data includes data described above, such as the AHI, an identification of one or more events experienced by the user, a number of events per hour, a pattern of events, a sleep score, a total sleep time, a total time in bed, a wake-up time, a rising time, a hypnogram, a total light sleep time, a total deep sleep time, a total REM sleep time, a number of awakenings, a sleep-onset latency, or any combination thereof.

Step 504 of the method 500 includes receiving second physiological data associated with the user during the second sleep session. The second physiological data can be the same as, or similar to, the first physiological data (step 502) described above except that the second physiological data represents the user's state during the second sleep session.

Step 505 of the method 500 includes determining a set of sleep-related parameters based on changes between the first physiological data and the second physiological data. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Step 506 of the method 500 includes determining one or more of a recommended therapy or recommended therapy settings based on the set of sleep-related parameters. According to some examples, the set of sleep-related parameters include one or more of a sleep score, a flow signal, a respiration signal, a respiration rate, an inspiration amplitude, an expiration amplitude, an inspiration-expiration ratio, a number of events per hour, a pattern of events, a sleep state, pressure settings of the respirator device, a heart rate, a heart rate variability, movement of the user, or any combination thereof. Other sleep-related parameters include oxygen saturation drops, average length of events, sympathetic nerve activity, subjective feedback, or any combinations thereof.

In some implementations, the recommended therapy for the user is to continue the use of one of the respiratory therapy systems described herein (e.g., a CPAP therapy system). The recommended therapy can be or includes, for example, adjusting a position of the user during a sleep session (e.g., encouraging the user to sleep on their side as opposed to on their back). Other recommended therapies can include a recommended bedtime or wakeup time for the user, medication, surgery, recommended diet and/or exercise (e.g., to aid in causing weight loss), or any combination thereof.

The recommended therapy is generally tailored to the user to aid in maximizing the efficacy of the therapy and aid in reducing or eliminating symptoms or generally improving sleep quality. In some cases, a physician may prescribe a certain therapy based on symptoms reported by the user and/or test results (e.g., from a sleep lab). For example, a physician might interpret the symptoms or test results and prescribe use of a CPAP system as therapy (e.g., if the user has an AHI over 15, the user is typically prescribed a CPAP system with no further analysis of the underlying data). However, because only certain information available to the physician, the prescribed therapy may not actually be the most effective therapy to treat the user (e.g., the user may be experiencing insomnia as opposed to apneas, or vice versa). Determining a recommended therapy based on the set of sleep-related parameters, which account for differences occurring during two distinct sleep session, can aid in identifying the most effective therapy for an individual user.

In some implementations of step 506, where the recommended therapy includes use of a respiratory therapy system (e.g., a CPAP system), the one or more recommended therapy settings can include a minimum pressure setting, a maximum pressure setting, or both. Generally, it is desirable to use the minimum pressure possible to sufficiently keep the airway open as higher pressures can potentially have a variety of adverse side effects (e.g., sore or dry throat, bloating, increased noise, etc.). Determining a recommended minimum and/or maximum pressure setting based on one or more of the sleep-related parameters can aid in optimizing and tailoring the pressure settings for the respiratory therapy system for that particular user.

The one or more recommended therapy settings can also include a recommended duration for the therapy. The recommended therapy duration can be a recommended duration for each sleep session (e.g., use the therapy for at least 6 hours each night) and/or a recommended duration over the course of multiple sleep sessions (e.g., use the therapy each night for at least one month). Generally, it is preferable that the user employ the respiratory therapy for every sleep session. However, in some cases, the user does not use the therapy every sleep session or for only a portion of the sleep session, especially if the user does not perceive a benefit from the therapy. Recommending a duration that is less than the entire sleep session can aid in encouraging the user to comply with the recommended therapy.

As described above, the recommended therapy and the recommended therapy settings are determined for the user based on the set of sleep-related parameters, which account for changes (or differences) in the user's physiological data occurring during two distinct sleep sessions. However, as more data accumulates over time, the recommended therapy or recommended therapy settings can be modified to be more tailored to the individual user. Thus, steps 503-506 can be repeated one or more times for multiple sleep sessions (e.g., two sleep sessions, five sleep sessions, thirty sleep sessions, one-hundred sleep sessions, etc.) to tailor the therapy type or therapy settings.

For example, if the determined recommended therapy (step 506) was to use a respiratory therapy system and the recommended therapy settings for that therapy included a maximum pressure setting and a minimum pressure setting for the respiratory therapy system, these pressure settings can be modified (e.g., increased or decreased) during step 503 based on increased or decreased efficacy of the therapy. As another example, if the determined recommended therapy (step 506) was to use a respiratory therapy system and the recommended therapy settings included a first user interface type (e.g., a nasal pillow), a subsequent use of step 503 can include modifying the recommended therapy settings to include a second user interface type (e.g., a full face mask) that is different than the first user interface type to aid in increasing the efficacy of the therapy (e.g., the recommended therapy of step 506 may reflect that the user is a mouth-breather, and thus the full face mask would be advantageous compared to the nasal pillow for that particular user).

As a further example, step 506 can include modifying the recommended therapy type. For example, if the modified therapy settings during step 503 include using a respiratory therapy system, step 506 can include modifying the recommended therapy to a different type of therapy (e.g., no use of the respiratory therapy system). For example, the first physiological data associated with the first sleep session may suggest that the user is experiencing apnea symptoms. However, upon collection of the second physiological data (step 504), the changes between the first and second physiological data may reveal that the user's symptoms are being caused by insomnia rather than an apnea. Additionally, by comparing the first physiological data (e.g., a rate of sleep apnea events) with the second physiological data (e.g., a body position), it may be determined that there is a correlation between the first and second physiological data. For example, the body position corresponds to a rate of sleep apnea events, which indicates that the user has a specific condition (e.g., positional sleep apnea) that may be treatable by alternative therapy systems or devices. The alternative therapy systems or devices, include using, for example, a mandibular repositioning device (MRD) system instead of a CPAP system, any other positional sleep apnea therapy device, a surgical procedure, etc. Optionally, recommendations of complementary devices (e.g., bedding, sleep blankets, pillows, etc.) are provided to the user for improving sleep quality based on the determined correlation between the first and second physiological data.

In further addition or alternative to the alternative therapy systems or devices described above, other therapy modes may be used to treat specific conditions of the user. For example, in some embodiments a determination is made of a particular disease phenotype, such as positional sleep apnea. In response to the determination, therapy settings are adjusted for the therapy mode according to the user position.

According to one example of therapy settings adjustment, a pressure range is increased. According to another example, a therapy setting increases a magnitude of the response to an event. According to yet another example, a therapy setting increases a response time of an auto-setting CPAP, when the user is on his/her back, compared to when the patient is on his/her side.

In alternative embodiments, one or more parameters of a therapy mode are learned over time. For example, the therapy mode is personalized to the user and the user's specific situation, e.g., the user sleeping position. According to a specific example, a first personalized therapy mode is enacted when the user is on his/her side, and a second (different) personalized therapy mode is enacted when the user is spine.

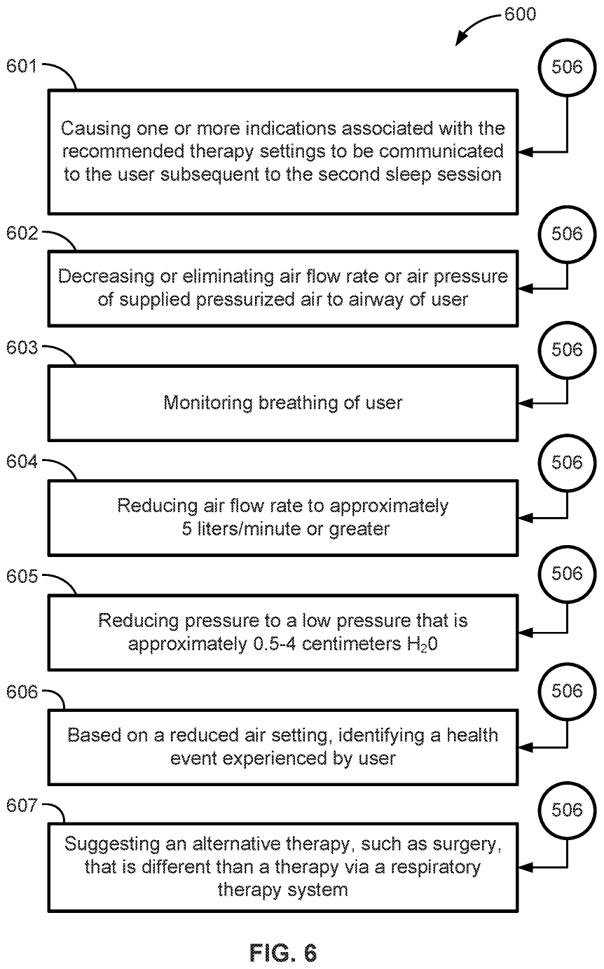

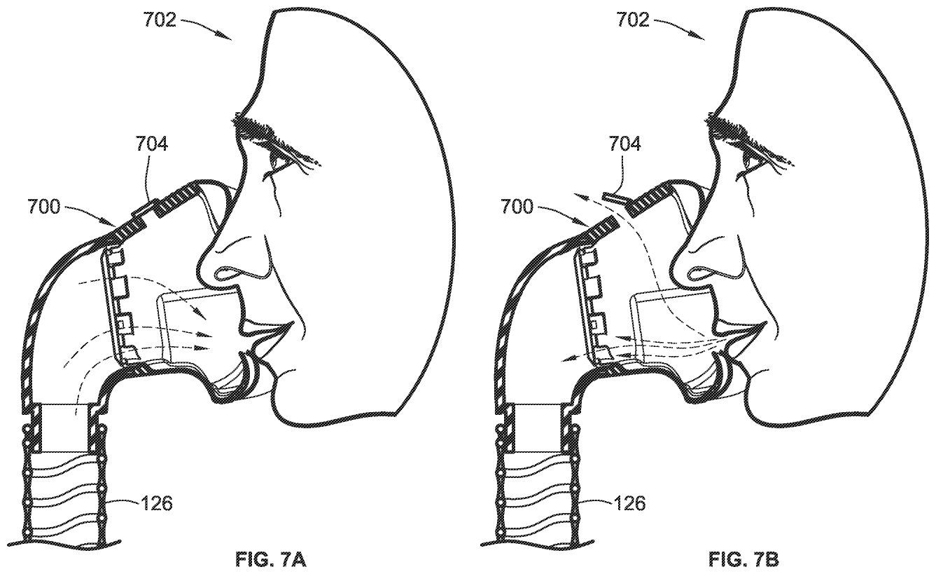

Referring to FIG. 6, various optional steps are for use with the method of 500. Step 601 includes causing one or more indications, which are associated with the recommended therapy settings, to be communicated to the user subsequent to the second sleep session. The one or more indications are communicated to the user, for example, via the user device 170 (e.g., via the display device 172). Additionally or alternatively, the one or more indications are communicated to a physician or other medical professional or provider.

According to one example, the one or more indications includes a recommendation that encourages the user to continue use of the respiratory system, a recommendation to discontinue use of the respiratory therapy system, a representation showing a health improvement of the user over time. According to a more specific example, the health improvement includes a decrease in apnea rates over time as an indicator of therapy success.

According to another example, a health score is provided to indicate the health of the user. The health score is generally determined based on the set of sleep-related parameters, objectively determined based on the set of sleep-related parameters, subjectively determined based on input received from the user, or any combination thereof. For example, the health score is any of the scores described above, including the MSLT score, the PSQI score, the self-reported subjective sleep score, myAir score, etc.

Step 602 includes decreasing or eliminating air flow rate or air pressure of supplied pressurized air to the airway of the user. The decreased or eliminated air flow rate or air pressure, as discussed above, are examples of modified therapy settings that are applied in step 503.

Step 603 includes monitoring the breathing of the user. The monitoring of the breathing is an additional step that is facilitated, for example, in response to applying the modified therapy settings at step 503. For example, in response to decreasing or eliminating air flow rate, the user's own breathing can now be monitored for evaluating how supplied pressurized air affects the user. If positive changes occur in the physiological data of the user based on the monitored breathing (e.g., the user is breathing better before decreasing air flow rate), a conclusion might be that the present therapy is providing improved results and should be strongly encouraged. However, if negative changes occur in the physiological data of the user based on the monitored breathing (e.g., the user is breathing better after decreasing air flow rate), a conclusion might be that the present therapy should be changed. Moreover, if no changes occur in the physiological data of the user based on the monitored breathing (e.g. the user is breathing the same before and after decreasing air flow rate), a conclusion might be that the present therapy does not provide any effect on the user and may be discontinued.

Step 604 includes reducing the air flow rate to approximately 5 liters/minute or greater. This reduction of air flow rate is an example of a modified therapy setting applied in step 503.

Step 605 includes reducing air pressure to a low pressure that is in the range of approximately 0.5-4 centimeters H₂O. This reduction of air pressure is another example of a modified therapy setting applied in step 503.

According to a specific example, Step 606 includes identifying a health event experienced by the user, based on a reduced air setting. The reduced air setting, for example, includes the reduction of air flow rate in step 604 and/or the reduce air pressure in step 605. According to some examples, the health event includes one or more of snoring, apnea, central apnea, obstructive apnea, mixed apnea, hypopnea, a mask leak, a restless leg, a sleeping disorder, choking, labored breathing, an asthma attack, an epileptic episode, a seizure, a cardiac arrhythmia, a cerebrovascular accident, or any combination thereof.

Step 607 includes suggesting an alternative therapy, such as surgery, that is different than a therapy via a respiratory therapy system. The alternative therapy is the recommended therapy described in step 506. The respiratory therapy system, for example, is the respiratory system 120 illustrated in FIG. 2.

Referring to FIGS. 7A and 7B, a mask 700 is placed on a user 702 for directing pressurized air to the airway of the user 702. The mask 700 is similar or identical to the user interface 124 illustrated in FIG. 2, and it includes a vent 704 for eliminating air when accumulated air in the mask 700 surpasses a predetermined maximum threshold. Optionally, a plurality of vents 704 are included in the mask 700 for providing additional outlets for eliminating air from the mask 700.

The vent 704 is movable between a closed position (illustrated in FIG. 7A) and an open position (illustrated in FIG. 7B). For example, the vent 704 is in the closed position during the first sleep session of step 502 and in the open position during the second sleep session of step 503. The vent 704 moves to the open position in response to pressurized air being reduced below a predetermined minimum threshold. By way of example, the predetermined minimum threshold is in the range of approximately 0.5-4 centimeters H₂O.

According to another example, the vent 704 moves to the open position in response to a predetermined level of carbon dioxide accumulating in the mask 700. In a specific example, the predetermined level of carbon dioxide is at least about 20%.

According to yet another example, the vent 704 moves to the closed position when the user 702 inhales (illustrated in FIG. 7A) and to the open position when the user 702 exhales (illustrated in FIG. 7B). In yet another example, the vent 704 moves to the open position in response to an air flow rate that is approximately 5 L/min or less.

Optionally, the vent 704 moves at about 2 centimeters H₂O to open to the atmosphere. For example, the vent 704 is a piece of silicon that pops open at low pressures. As the user takes a breath in (FIG. 7A), the vent 704 closes to the atmosphere and the pressure of about 2 centimeters H₂O keeps the vent 704 closed. If the pressure drops below about 2 centimeters H₂O, the vent 704 pops back open.

The mask 700 has a mask orientation that is sensed by one or more of the sensors 130. The mask orientation is included in one or more of the first physiological data or the second physiological data of steps 502 and 504. Alternatively or additionally, a body position of the user 702 is sensed by one or more of the sensors 130. The body position is included in one or more of the first physiological data or the second physiological data of steps 502 and 504. Based on the sensed changes in the body position and/or the mask orientation, and further based on air flow, an algorithm determines whether the user has positional sleep apnea. The algorithm may also determine the optimal therapy settings (e.g., reduced or increased pressure) based on the sensed position of the user. As described above, the sensed body position is correlated to a specific condition (e.g., positional sleep apnea) for which the optimal therapy settings are, then, determined.

According to a specific example, supplied pressurized air to the user is ramped up from a starting point of approximately 4-6 centimeters H₂O. The resulting air flow is dependent on the specific mask, but a general standard provides for no more than 20% CO₂ in the mask, with a flow rate of approximately 20 L/min (which can go up to approximately 50 L/min). This is the device flow, i.e., the flow supplied by the respiratory therapy system. However, a vent flow is a mixture of air flow from the user and the device flow. The average vent flow is generally equal to the average device flow, unless there's an air leak. The vent has a maximum vent flow, with the device flow handling the majority of that maximum vent flow, but not all of that it. As such, there's a portion of the air leftover so that when the user exhales some of the exhaled air remains in the mask. To help eliminate any leftover exhaled air, which causes the accumulation of CO₂ in the mask, the low flow rate mentioned above is desired.

When controlling the air flow from the flow generator (e.g., the respiratory device 122 of FIG. 2), an estimate of user air flow is measured by a pressure sensor (e.g., the pressure sensor 132 of FIG. 1) from the user's breathing in and out. Thus, this estimate helps determine the appropriate low flow rate to avoid accumulation of CO₂ in the mask. This is beneficial because the user's drive to breath is driven by the user's blood CO₂level, which as it increases causes the user's breathing to increase. In turn, this increase in the user's breathing can cause an apnea occurrence. Conversely, if the blood CO₂ level is too low, the user can experience a central sleep apnea occurrence in which the brain's user has the user not breathing. Accordingly, maintaining the appropriate level of CO₂ is beneficially controlled at least in part using one or more vents in the mask, as discussed above.

One or more elements or aspects or steps, or any portion(s) thereof, from one or more of any of claims 1-59 below can be combined with one or more elements or aspects or steps, or any portion(s) thereof, from one or more of any of the other claims 1-59 or combinations thereof, to form one or more additional implementations and/or claims of the present disclosure.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present invention have been described above, it should be understood that they have been presented by way of example only, and not limitation. Numerous changes to the disclosed embodiments can be made in accordance with the disclosure herein, without departing from the spirit or scope of the invention. Thus, the breadth and scope of the present invention should not be limited by any of the above described embodiments. Rather, the scope of the invention should be defined in accordance with the following claims and their equivalents.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

1. A method comprising:
applying, via a respiratory therapy system, initial therapy settings for a user during a first sleep session in which the user uses the respiratory therapy system;
receiving, from one or more sensors, first physiological data generated during the first sleep session;
applying, via the respiratory therapy system, modified therapy settings during a second sleep session of the user, the second sleep session being subsequent to the first sleep session, the modified therapy settings being different than the initial therapy settings;
receiving, from the one or more sensors, second physiological data generated by the one or more sensors during the second sleep session;
determining a set of sleep-related parameters based on changes between the first physiological data and the second physiological data; and
determining one or more of a recommended therapy or recommended therapy settings based on the set of sleep-related parameters.

2. The method of claim 1, further comprising causing one or more indications associated with the recommended therapy settings to be communicated to the user, via a user device, subsequent to the second sleep session.

3. The method of claim 2, wherein the one or more indications include a recommendation that encourages the user to continue use of the respiratory therapy system.

4. The method of claim 2, wherein the one or more indications include a recommendation to discontinue use of the respiratory therapy system.

5-10. (canceled)

11. The method of claim 1, wherein the modified therapy settings include a reduced air setting that decreases or eliminates air flow rate or air pressure of supplied pressurized air to the airway of the user.

12-16. (canceled)

17. The method of claim 1, wherein the user interface is in the form of a mask having a vent, the vent eliminating air when accumulated air in the mask surpasses a predetermined maximum threshold.

18. The method of claim 17, wherein the vent is in a closed position during the first sleep session, the vent being in an open position during the second sleep session.

19. The method of claim 18, further comprising moving the vent to the open position in response to the pressurized air being reduced below a predetermined minimum threshold.

20. The method of claim 19, wherein the predetermined minimum threshold is in the range of approximately 0.5-4 centimeters H₂O.

21. The method of claim 18, further comprising moving the vent to the open position in response to a predetermined level of carbon dioxide accumulating in the mask.

22. The method of claim 21, wherein the predetermined level of carbon dioxide being at least about 20%.

23. The method of claim 18, further comprising:
moving the vent to the closed position when the user inhales; and
moving the vent to the open position when the user exhales.

24. The method of claim 18, further comprising moving the vent to the open position in response to an air flow rate being approximately 5 liters/minute or less.

25-26. (canceled)

27. The method of claim 1, wherein the first physiological data and the second physiological data include a body position of the user.

28-31. (canceled)

32. The method of claim 1, further comprising:
determining the recommended therapy based on a positional sleep apnea; and
adjusting the recommended therapy settings according to a position of the user.

33. The method of claim 32, wherein the recommended therapy settings include at least one of (a) increasing a pressure range, (b) increasing a magnitude of a response to a health event, or (c) increasing a response time of an auto-setting for a continuous positive airway pressure (CPAP) system when the user changes between a first position and a second position.

34. The method of claim 33, wherein first position is a back position and the second position is a supine position.

35. The method of claim 32, further comprising personalizing the recommended therapy and/or the recommended therapy settings based on the position of the user.

36-39. (canceled)

40. A system comprising:
a respiratory therapy system including:
a respiratory device configured to supply pressurized air; and
a user interface coupled to the respiratory device via a conduit, the user interface being configured to engage a user and aid in directing the supplied pressurized air to an airway of the user;
a memory storing machine-readable instructions; and
a control system including one or more processors configured to execute the machine-readable instructions to:
apply initial therapy settings for the user during a first sleep session in which the user uses the respiratory therapy system;
receive first physiological data generated by one or more sensors during the first sleep session;
apply modified therapy settings during a second sleep session of the user, the second sleep session being subsequent to the first sleep session, the modified therapy settings being different than the initial therapy settings;
receive second physiological data generated by the one or more sensors during the second sleep session;
determine a set of sleep-related parameters based on changes between the first physiological data and the second physiological data; and
determine recommended therapy settings based on the set of sleep-related parameters.

41-45. (canceled)

46. The system of claim 40, wherein the user interface is in the form of a mask having a vent, the vent eliminating air when accumulated air in the mask surpasses a predetermined maximum threshold.

47. The system of claim 46, wherein the vent is in a closed position during the first sleep session, the vent being in an open position during the second sleep session.

48. The system of claim 46, wherein the vent moves to the open position in response to the pressurized air being reduced below a predetermined minimum threshold.

49. The system of claim 48, wherein the predetermined minimum threshold is in the range of approximately 0.5-4 centimeters H₂O.

50. The system of claim 46, wherein the vent moves to the open position in response to a predetermined level of carbon dioxide accumulating in the mask.

51. The system of claim 50, wherein the predetermined level of carbon dioxide being at least about 20%.

52-59. (canceled)
